(19) United States
(12) Patent Application Publication
(10) Pub. No.: US 2013/0245575 A1
(43) Pub. Date: Sep. 19, 2013

(54) BODY PRODUCT AND METHOD OF DELIVERY

(71) Applicant: Sara Jo Griffin, Hudson, WI (US)

(72) Inventor: Sara Jo Griffin, Hudson, WI (US)

(21) Appl. No.: 13/607,432

(22) Filed: Sep. 7, 2012

Related U.S. Application Data
(60) Provisional application No. 61/685,272, filed on Mar. 14, 2012.

(51) Int. Cl. A61K 36/185; A61K 36/886; A61K 36/53; A61K 31/355; A61K 31/47; A61K 31/4453; A61K 31/5377; A61K 36/14; A61K 36/889; A61K 33/22; A61K 36/71; A61K 31/4425; A61P 15/02; A61P 23/00; A61P 25/04; A61P 31/00; A61M 35/00; A61K 36/28

(52) U.S. Cl. 604/290; 424/764; 424/769; 424/744; 424/745; 514/458; 514/312; 514/317; 514/239.2; 424/727; 424/725; 424/659; 424/726; 514/358; 604/304

(57) ABSTRACT

A composition and method for administering topical and external topical and external body products for prevention and treatment of anal/vaginal ailments, the composition comprising a mixture of a melting agent, a healing agent, a hardening agent a moisture barrier agent, a carrier agent, the mixture in solid state and undergoing a phase change to a lubricating state when applied to a human body.

### CROSS REFERENCE TO RELATED APPLICATIONS

This application claims priority to currently pending U.S. Provisional Application Ser. No. 61/685,272; filed on Mar. 14, 2012; titled DELIVERY METHOD OF TOPICAL AND EXTERNAL BODY PRODUCTS.

### FIELD OF THE INVENTION

This invention relates to a delivery system for the application of topical and external body products and, more specifically a topical and external composition and method for administering topical and external topical and external body products for prevention and treatment of anal/vaginal ailments, such as diaper rash and hemorrhoids, in a fast no-mess manner while reducing or preventing the risk of cross-contamination.

### STATEMENT REGARDING FEDERALLY SPONSORED RESEARCH OR DEVELOPMENT

None

### REFERENCE TO A MICROFICHE APPENDIX

None

### BACKGROUND OF THE INVENTION

Currently, topical and external body products on the market that cater to prevention and treatment of anal/vaginal ailments or for children, adults, and elderly persons are generally made in forms of creams, salves, and ointments. It should be noted that topical and external body products, as defined hereinafter, are a category of body products that are specifically design to be use only on the exterior surfaces of the body in treating ailments such as various type of rash and hemorrhoids especially in the regions of the body human body that may be more susceptible to cross-contamination anal area and vaginal area. These products have inherent viscous properties and therefore are messy in application and involve necessary clean-up by the person applying the product after each use. The topical and external products that are available to date typically involve an application method that is inconvenient and extremely unhygienic to all people involved. In instances where a person comes in contact with any part of the anal/vaginal area, the risk of cross-contamination is very high.

There thus is a need for a composition and method for administering topical and external topical and external body products for prevention and treatment of anal/vaginal ailments in a fast no-mess manner while reducing or preventing the risk of cross-contamination between multiple users and between person(s) applying the product.

### BRIEF SUMMARY OF THE INVENTION

The present invention comprises a composition and a sanitary method for administering topical and external topical and external body products for prevention and treatment of anal/vaginal ailments, the composition comprising a mixture of a melting agent, a healing agent, a hardening agent, a moisture barrier agent, and a carrier agent, with the mixture sandwiched between a first and second protective layer and in solid state and undergoing a phase change to a lubricating state when applied to a human body.

The sanitary method of administering topical and external topical and external body products for the prevention and treatment of anal/vaginal ailments comprising the steps of selecting a single dose of anal/vaginal treatment composition, peeling a first protective layer from the single dose of anal/vaginal treatment composition to expose a body-treating surface while maintaining a second protective layer on the single dose of anal/vaginal treatment composition, applying the body-treating surface of the single dose of anal/vaginal treatment composition to the receiver's body while preventing the user's fingers from coming into contact with the receiver's skin, and maintaining the single dose of anal/vaginal treatment composition to the receiver's body to trigger a phase change of the single dose of anal/vaginal treatment composition from a solid state to a liquid state.

### BRIEF DESCRIPTION OF THE DRAWINGS

FIG. 1 shows a top view of an embodiment of an anal/vaginal treatment composition of the present invention in individually wrapped single dose form;

FIG. 2 shows a side view of the individually wrapped single dose anal/vaginal treatment composition of FIG. 1; and

FIG. 3 shows a side view of an embodiment of an anal/vaginal treatment composition in roll form.

### DESCRIPTION OF THE PREFERRED EMBODIMENT

The present invention comprises a composition for administering topical and external body products for the prevention and treatment of anal/vaginal ailments. The composition generally comprises a mixture of a melting agent, a healing agent, a hardening agent, a moisture barrier agent, and a carrier agent wherein the composition is in solid form and changes to liquid form when applied to the human body. Although the various agents for the composition preferably comprises organic ingredients, synthetic ingredients, or a mixture of both may be used to create a formula for the composition.

Examples of some of the possible but non-limiting organic ingredients that may be used for the agents of the above composition includes the melting agent selected from the group comprising of Coconut Oil, Cocoa Butter, and Shea Butter. The healing agent of the composition selected from the group comprising of Calendula, Shea Butter, Sunflower oil, Aloe Vera, Lavender Oil, and Vitamin E. The hardening agent of the composition selected from the group comprising of Beeswax, Coconut Oil, and Shea Butter. The moisture barrier agent of the composition selected from the group comprising of Aluminum Hydroxide Gel, Cocoa butter, glycerin, Zinc Oxide, Coconut Oil, Acid Mantle, Petroleum, Shark Liver Oil, Lanolin, Mineral Oil, White Petroleum, Starch, Cod Liver Oil, and Kaolin. The carrier agent of the composition selected from the group comprising of Soybean Oil, Canola Oil, Beeswax, Jojoba Oil, Sweet Almond Oil, Olive Oil, and Sesame Seed Oil.

The composition for administering topical and external topical and external body products may further include an anesthetics agent, an analgesics agent, an antimicrobial agent, a pH balancing agent, and other related agents. Examples of the aforementioned include but are not limited to the anesthetics agent of the composition selected from the group comprising of Benzocaine, Benzyl Alcohol, Dibucaine, Dyclonine, Lidocaine, Pramoxine, and Tetracaine. The analgesics agent of the composition selected from the group comprising of Menthol, Camphor, Juniper Tar, Rosemary Oil, and Chamomile. The antimicrobial agent of the composition selected from the group comprising of Coconut Oil, Jojoba Oil, Boric Acid, Hydrastis, Phenol, Benzalkonium Chloride, Cetylpyridinium Chloride, Benzethonium Chloride, and Resoricol. An example of the pH-balancing agent includes but is not limited to Lavender Oil.

Referring now specifically to the drawings, in which identical or similar parts are designated by the same reference numerals throughout, a detailed description of the present invention is given. It should be understood that the following detailed description relates to the best presently known embodiment of the invention. However, the present invention can assume numerous other embodiments, as will become apparent to those skilled in the art.

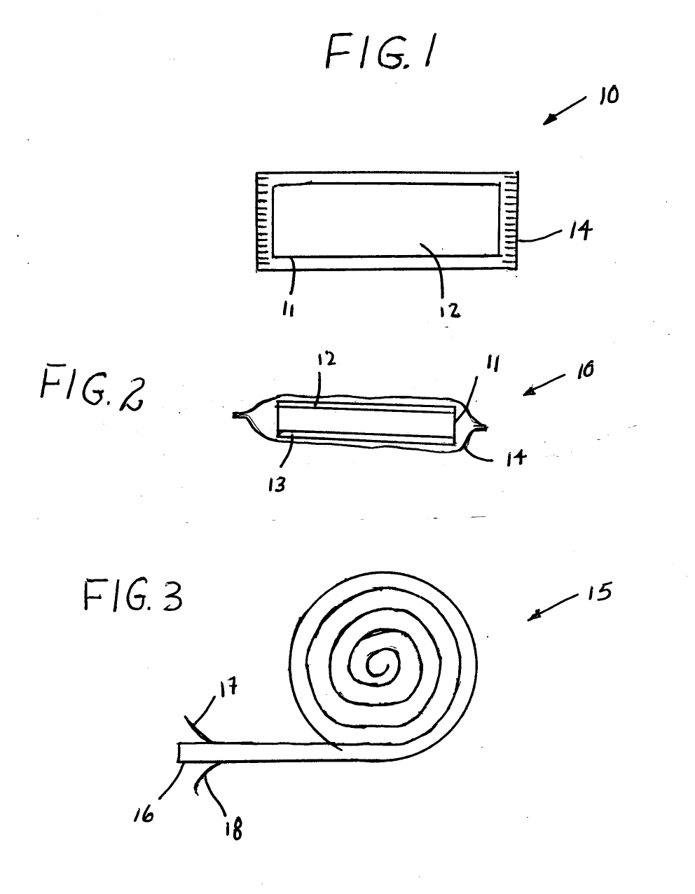

Now referring to FIGS. 1,2, and 3, FIG. 1 shows a top view of an embodiment of an anal/vaginal treatment composition of the present invention. FIG. 2 shows a side view of the anal/vaginal treatment composition 10 of FIG. 1. Anal/vaginal treatment composition 10 generally comprises a single dose 11 of the anal/vaginal treatment composition sandwiched between a first protective layer 12 and a second protective layer 13. Protective layers 12 and 13 may be composed of a variety of materials that are attachable and yet cleanly peelable from the treatment composition. Examples of possible materials for protective layers 12 and 13 include but are not limited to wax paper, plastic wrap, parchment paper, and aluminum foil.

A feature of the present invention is that treatment composition 10 is preferably maintained in solid form until treatment composition 10 comes into contact with a human body. Upon contact onto the intended area, a phase change of treatment composition 10 from a solid state to a lubricating state is triggered by temperature or pressure or a combination thereof. The lubricating state is defined as a state in which treatment composition 10 may be spreadable on the intended area of the body and ranging from a cream, salve or ointment to a free flowing liquid and anywhere in between.

As shown in the embodiment of FIGS. 1 and 2, single dose 11 is individually wrapped with a plastic wrapping container 14 or the like for single use application. Preferable, each individually wrapped piece of single dose 11 is designed and sealed to contain the single dose 11 in a way that if the single dose 11 gets warmed and melts such as due to shipping circumstances or improper storage, single dose 11 may regain its solid state and original shape because of the manner in which single dose 11 is packaged as soon as the surrounding temperature falls within a prescribed range, preferable a range that is lower than body temperature.

In regards to the size of single dose 11, the size of single dose 11 may vary according to the age range, size, and body type of the receiver and the purpose single dose 11 is designated for. For example, the size of single dose 11 may be smaller for a children's' diaper rash product and larger for an adults' anal/vaginal irritation. Although it should be construed as limiting, an example for the sizes of single dose 11 may comprise a length ranging between 0.01 to 12 inches, a width ranging between 0.01 to 12 and a thickness ranging between 0.01 to 8 inches. It is anticipated that treatment composition 10 may be sold in a container containing a plurality of individually wrapped single dose 11 of varying sizes to treat multiple individuals of varying ages and sizes.

FIG. 3 shows a side view of an embodiment of an anal/vaginal treatment composition comprising a roll 15 containing multiple dosage of anal/vaginal treatment composition 16. Multiple dosage of anal/vaginal treatment composition 16 is sandwiched between a first protective layer 17 and a second protective layer 18. Similar to protective layers 12 and 13, protective layers 17 and 18 may be composed of a variety of materials that are attachable and yet cleanly peelable from the treatment composition. Examples of possible materials for protective layers 17 and 18 include but are not limited to wax paper, plastic wrap, parchment paper, and aluminum foil. Roll 15 allows a user to customize a single dose of anal/vaginal treatment composition 16 for treatment on a receiver based on the measurements of the receiver's body.

For example, in customizing a single dose of anal/vaginal treatment composition 16 the user may first visually estimate a suitable size or amount of anal/vaginal treatment composition 16 required for a single dose on a particular receiver based trial and error, product pre-measurement instructions or the user's prior experience. Once the suitable size or amount of anal/vaginal treatment composition 16 has been determined, the user may either tear or cut the desired amount from roll 15 and use accordingly.

Referring back to FIGS. 1 and 2, in administering treatment composition 10, the user first peels first protective layer 12 from the single dose of anal/vaginal treatment composition 11 to expose a body-treating surface while maintaining a second protective layer 13 on the single dose of anal/vaginal treatment composition 11. The user then applies the body-treating surface of the single dose of anal/vaginal treatment composition 11 to the receiver's body while preventing the user's fingers from coming into contact with the receiver's skin. The single dose of anal/vaginal treatment composition 11 is then engaged to the receiver's body to trigger a phase change of the single dose of anal/vaginal treatment composition 11 from a solid state to a lubricating state to treat the desired region of the receiver's body. It should be noted that once in the lubricating state single dose of anal/vaginal treatment composition 11 may be self-spreading by movement of the receiver's buttock cheeks thereby reducing the work required by the user in the application of single dose of anal/vaginal treatment composition 11 to the receiver's body.

In regards to the second protective layer 13, the user may use the second protective layer 13 to apply the body-treating surface of the single dose of anal/vaginal treatment composition 11 to the receiver's body to prevent the user's fingers from coming into contact with the receiver's skin or with single dose of anal/vaginal treatment composition 11. The user may optionally peel the second protective layer 13 from the single dose of anal/vaginal treatment composition 11 and then apply the single dose of anal/vaginal treatment composition 11 to the receiver's body. Although the user's fingers will be touching single dose of anal/vaginal treatment composition 11, this method is still sanitary since the user's fingers would only be touching the topside of single dose of anal/vaginal treatment composition 11 and will not come from into contact with the receiver's body part.

Since the user's fingers rarely if ever come in direct contact with the skin of the receiver's body part including the receiver's anal/vaginal area, the present invention makes the interaction and application of body product(s) more hygienic and more convenient due to the lack of a runny, sticky mess that such products are known to create.

In the individually pre-wrapped single dose form each application is intended and packaged for a single use. Each individually wrapped piece of single dosage of treatment composition is designed and sealed to contain the product in a way that if the body product did in fact get warm and melts, the product does not leak. The individually wrapped piece of treatment composition may also be sized and package to discreetly be carried in a female's purse and various diaper bags and carry-a-longs.

An example of the steps of making the composition for administering topical and external topical and external body products of the present comprising the steps of boiling 2-3 inches of water in a sauce pan and placing a metal bowl inside boiling water. The ingredient with highest melting point, in this instance Beeswax, is then added into the metal bowl. After the beeswax has melted Coconut oil is then added into the metal bowl and stirred until the beeswax and oils are combined.

Once the beeswax and coconut oil are in a liquid state, Sweet Almond Oil, Jojoba Oil, Lavender Oil, Calendula Oil, Vitamin E, Zinc Oxide are then added to the metal bowl. The ingredients are then stirred in the metal until all of the ingredients are blended.

Once blended, the composition is then transferred onto flat wax paper or the like and formed into a plurality of single dosages that are roughly 1.5″ wide and 2″ long. The single dosages of composition are then allowed to harden such as by placing them in a hardening in a freezer for 5-10 minutes.

After the single dosages of composition have harden and cooled the wax paper is then peeled away and the single dosages of composition may be stored in a hard-shelled container with wax paper in between each piece at room temperature.

The present invention also includes a sanitary and no-mess method of administering topical and external topical and external body products for the prevention and treatment of anal/vaginal ailments for people in all age ranges. A feature of the present invention is that by using the aforementioned method of delivery, the user's fingers rarely if ever come in direct contact with the skin of the receiver's anal/vaginal area, making the interaction and application of body product hygienic and very convenient due to the lack of a runny, sticky mess that viscous products create. The method generally comprises the steps of (1) selecting a single dose of anal/vaginal treatment composition 11 sandwiched between a pair of protective layers 12, 13; (2) peeling a first protective layer 12 from the single dose of anal/vaginal treatment composition 11 to expose a body-treating surface while maintaining a second protective layer 13 on the single dose of anal/vaginal treatment composition 11; (3) applying the body-treating surface of the single dose of anal/vaginal treatment composition 11 to the receiver's body while preventing the user's fingers from coming into contact with the receiver's skin; and (4) maintaining the single dose of anal/vaginal treatment composition 11 to the receiver's body to trigger a phase change of the single dose of anal/vaginal treatment composition 11 from a solid state to a lubricating state.

The above method may further include the steps of (6) selecting a single dose of anal/vaginal treatment composition 11 comprising a melting agent, a healing agent, a hardening agent, a moisture barrier agent, and a carrier agent in the solid state; (7) peeling the second protective layer 13 from the single dose of anal/vaginal treatment composition 11 and then applying the body-treating surface of the single dose of anal/vaginal treatment composition 11 to the receiver's body while preventing the user's fingers from coming into contact with the receiver's skin; (8) using the second protective layer 13 to apply the body-treating surface of the single dose of anal/vaginal treatment composition 11 to the receiver's body while preventing the user's fingers from coming into contact with the receiver's skin; (9) obtaining an individually pre-wrapped dose of anal/vaginal treatment composition 11; and (10) customizing a single dose of anal/vaginal treatment composition from a roll of anal/vaginal treatment composition based on the measurements of the receiver's body.

I claim:

1. A composition for administering topical and external topical and external body products for the prevention and treatment of anal/vaginal ailments, the composition comprising a mixture of a melting agent, a healing agent, a hardening agent, a moisture barrier agent, a carrier agent; said mixture in solid state and undergoing a phase change to a lubricating state when applied to a human body.

2. The composition for administering topical and external topical and external body products of claim 1 wherein said melting agent is selected from the group consisting of Coconut Oil, Cocoa Butter, and Shea Butter.

3. The composition for administering topical and external topical and external body products of claim 1 wherein said healing agent is selected from the group consisting of Calendula, Shea Butter, Sunflower oil, Aloe Vera, Lavender Oil, and Vitamin E.

4. The composition for administering topical and external topical and external body products of claim 1 wherein said hardening agent is selected from the group consisting of Beeswax, Coconut Oil, and Shea Butter.

5. The composition for administering topical and external topical and external body products of claim 1 wherein said moisture barrier agent is selected from the group consisting of Aluminum Hydroxide Gel, Cocoa butter, glycerin, Zinc Oxide, Coconut Oil, Acid Mantle, Petroleum, Shark Liver Oil, Lanolin, Mineral Oil, White Petroleum, Starch, Cod Liver Oil, and Kaolin.

6. The composition for administering topical and external topical and external body products of claim 1 wherein said carrier agent is selected from the group consisting of Soybean Oil, Canola Oil, Beeswax, Jojoba Oil, Sweet Almond Oil, Olive Oil, and Sesame Seed Oil.

7. The composition for administering topical and external topical and external body products of claim 1 further including an anesthetics agent selected from the group consisting of Benzocaine, Benzyl Alcohol, Dibucaine, Dyclonine, Lidocaine, Pramoxine, and Tetracaine.

8. The composition for administering topical and external topical and external body products of claim 1 further including an analgesics agent selected from the group consisting of Menthol, Camphor, Juniper Tar, Rosemary Oil, and Chamomile.

9. The composition for administering topical and external topical and external body products of claim 1 further including an antimicrobial agent selected from the group consisting of Coconut Oil, Jojoba Oil, Boric Acid, Hydrastis, Phenol, Benzalkonium Chloride, Cetylpyridinium Chloride, Benzethonium Chloride, and Resoricol.

10. The composition for administering topical and external topical and external body products of claim 1 further including a pH-balancing agent.

11. The composition for administering topical and external topical and external body products of claim 1 wherein said mixture is sandwiched between a first protective layer and a second protective layer.

12. The composition for administering topical and external topical and external body products of claim 11 wherein a single dosage of said composition is in individually pre-wrapped form.

13. The composition for administering topical and external topical and external body products of claim 11 wherein said composition is in roll form to allow for the customization of a single dose of said composition based on the measurements of the receiver's body.

14. The composition for administering topical and external topical and external body products of claim 1 wherein the phase change of said mixture when applied to a human body is triggered by temperature or pressure or a combination thereof.

15. A sanitary method of administering topical and external topical and external body products for the prevention and treatment of anal/vaginal ailments comprising the steps of:
selecting a single dose of topical and external anal/vaginal treatment composition sandwiched between a pair of protective layers;
peeling a first protective layer from said single dose of topical and external anal/vaginal treatment composition to expose a body-treating surface while maintaining a second protective layer on said single dose of topical and external anal/vaginal treatment composition;
applying said body-treating surface of said single dose of topical and external anal/vaginal treatment composition to the receiver's body while preventing the user's fingers from coming into contact with the receiver's skin; and
maintaining said single dose of topical and external anal/vaginal treatment composition to the receiver's body to trigger a phase change of said single dose of topical and external anal/vaginal treatment composition from a solid state to a lubricating state.

16. The sanitary method of administering topical and external topical and external body products of claim 15 including the step of peeling the second protective layer from said single dose of topical and external anal/vaginal treatment composition and then applying said body-treating surface of said single dose of topical and external anal/vaginal treatment composition to the receiver's body while preventing the user's fingers from coming into contact with the receiver's skin.

17. The sanitary method of administering topical and external topical and external body products of claim 15 including the step of using said second protective layer to apply said body-treating surface of said single dose of topical and external anal/vaginal treatment composition to the receiver's body while preventing the user's fingers from coming into contact with the receiver's skin.

18. The sanitary method of administering topical and external topical and external body products of claim 15 wherein the step of selecting a single dose of topical and external anal/vaginal treatment composition comprises obtaining an individually pre-wrapped dose of topical and external anal/vaginal treatment composition.

19. A composition for administering topical and external topical and external body products for the prevention and treatment of anal/vaginal ailments, the composition comprising a mixture of:
a melting agent selected from the group consisting of Coconut Oil, Cocoa Butter, and Shea Butter;
a healing agent selected from the group consisting of Calendula, Shea Butter, Sunflower oil, Aloe Vera, Lavender oil, and Vitamin E;
a hardening agent selected from the group consisting of Beeswax, Coconut oil, and Shea butter;
a moisture barrier agent selected from the group consisting of Aluminum Hydroxide gel, Cocoa butter, glycerin, Zinc Oxide, Coconut oil, Acid Mantle, Petroleum, Shark Liver oil, Lanolin, Mineral oil, White petroleum, Starch, Cod Liver oil, and Kaolin;
a carrier agent selected from the group consisting of Soybean oil, Canola oil, Beeswax, Jojoba oil, Sweet Almond oil, Olive oil, and Sesame Seed oil;
an anesthetics agent selected from the group consisting of Benzocaine, Benzyl Alcohol, Dibucaine, Dyclonine, Lidocaine, Pramoxine, and Tetracaine;
an analgesics agent selected from the group consisting of Menthol, Camphor, Juniper tar, Rosemary oil, and Chamomile;
an antimicrobial agent selected from the group consisting of Coconut oil, Jojoba oil, Boric acid, Hydrastis, Phenol, Benzalkonium Chloride, cetylpyridinium chloride, Benzethonium chloride, and Resoricol; and
a pH balancing agent;
said mixture sandwiched between a first protective and a second protective layer, said mixture is in solid state and changing to a lubricating state when applied to the human body.

20. The composition for administering topical and external topical and external body products of claim 19 wherein a single dosage of said composition is in individually pre-wrapped form.
